(19) United States
(12) Patent Application Publication
(10) Pub. No.: US 2013/0005026 A1
(43) Pub. Date: Jan. 3, 2013

(54) CERVICAL CANCER SCREENING BY MOLECULAR DETECTION OF HUMAN PAPILLOMAVIRUS-INDUCED NEOPLASIA

(75) Inventors: Peter GOMBRICH, Salinas, CA (US); Paul Vichi, Lincoln, VT (US)

(73) Assignee: Cermed Corporation, Monterey, CA (US)

(21) Appl. No.: 13/470,115

(22) Filed: May 11, 2012

Related U.S. Application Data
(63) Continuation-in-part of application No. PCT/US2010/056404, filed on Nov. 11, 2010.
(60) Provisional application No. 61/260,829, filed on Nov. 12, 2009.

(51) Int. Cl. C12M 1/42; G01N 27/327; C12M 1/34; B82Y 15/00

(52) U.S. Cl. 435/287.2; 204/403.04; 977/746; 977/920; 977/745; 977/750; 977/752

(57) ABSTRACT

Point-of-care tools for screening biological samples for markers associated with pathogenic microbial infections. In particular, devices and systems for screening cervical cells for the expression of proteins, which occur as a result of human papillomavirus infection and progression to invasive cervical cancer.

### CROSS-REFERENCE TO RELATED APPLICATIONS

This application is a Continuation-in-Part of PCT/US2010/056404, filed Nov. 11, 2010, which claims the benefit of U.S. Provisional Application No. 61/260,829, filed Nov. 12, 2009, each of which is hereby incorporated by reference, in its entirety.

### BACKGROUND

1. Field

This disclosure relates to point-of-care tools for screening biological samples for markers associated with pathogenic microbial infections. In particular, the present disclosure provides methods, devices and systems for screening cervical cells for the expression of proteins, which occur as a result of human papillomavirus infection and progression to invasive cervical cancer.

2. Related Art

According to a recent report from the World Health Organization, cervical cancer is the fifth most deadly cancer for women in the world. Cervical cancer screening is commonly based on cytological and colposcopic analyses. The generally accepted cytological smear of the cervix (Papanicolaou test or Pap smear) has led to a reduction in the incidences of and mortalities caused by cervical cancer. The common Pap smear detects cellular abnormalities and thus the development of potentially pre-cancerous lesions. In a Pap smear, the collected cells are placed on a glass slide, stained and examined by a specially-trained and qualified cytotechnologist using a light microscope. In short, it is a subjective analysis with several known disadvantages, such as an increase in false-negatives and equivocal results as a consequence of debris obscuring abnormal cells.

The single most important risk factor for development of cervical cancer is human papillomavirus (HPV) infection. Although over 100 strains of HPV have been identified, only a subset are classified as high-risk (16, 18, 31, 33, 35, 39, 45, 51, 52, 56, 58, 59, 68, 73, and 82) or probable high-risk (26, 53, and 66) types (Munoz et al., NEJM, 348:518-527, 2003). Of these HPV types, HPV16 and HPV18 are reported to cause nearly 70% of all cervical cancer cases.

HPV-induced cervical cancer involves the following steps: (1) initial HPV infection, (2) persistent HPV infection, (3) transforming HPV infection, in the presence or absence of integration of HPV DNA into the host cell genome, (4) development of precancerous lesions and (5) development of invasive cancer. Evidence of HPV infection is very common, especially in young women. However, HPV infections typically resolve on their own or are suppressed by the immune system without causing serious pathology (e.g., advanced cervical disease including cervical intraepithelial neoplasia 2 (CIN 2), CIN 3 and invasive cancer).

The HYBRID CAPTURE® 2 (hc2) High-Risk HPV DNA Test developed by Digene (now QIAGEN) is a nucleic acid hybridization assay, with signal amplification. The assay uses chemiluminescent detection of HPV DNA in a microplate format. According to the manufacturer, this HPV DNA test identifies the presence of 13 of the 15 high-risk HPV types, while other versions of the test also detect the presence of five low risk HPV types.

In summary, on its own, a Pap smear permits identification of abnormal cervical cells, but not HPV infection. On the other hand, the HPV DNA tests permit the identification of HPV infection but not abnormal cervical cells. Thus neither test by itself is sufficient for detecting both infection with high-risk HPV strains and the presence of precancerous cells. Given these limitations, there is a pressing need for a single, non-morphological test that can address whether an HPV infection is causing cancerous changes to a host cell. Ideally this test would be in the form of a quick, disposable, point-of-care, molecular, cervical cancer screening system. The disposability and point-of-care aspects would not necessitate a laboratory infrastructure and as such would permit the test to be utilized globally. The present disclosure satisfies these needs.

### SUMMARY

This disclosure relates to point-of-care tools for screening biological samples for markers associated with pathogenic microbial infections. In particular, the present disclosure provides methods, devices and systems for screening cervical cells for the expression of proteins that occur because of human papillomavirus infection and progression to invasive cervical cancer.

In one embodiment of a system for cervical cancer screening, the system includes a flow cytometric platform having a micro-electro-mechanical system (MEMS) chip embedded within a cartridge; and reagents disposed within said cartridge. The reagents include at least one HPV-reactive reagent; and at least one neoplastic biomarker-reactive reagent. The flow cytometric platform of the embodiment is adapted to detect binding of the HPV-reactive reagent to HPV and binding of the neoplastic biomarker-reactive reagent to a neoplastic biomarker, if HPV and/or the neoplastic biomarker are present in a sample that includes cervical cells. In this embodiment, detection of elevated levels of HPV and a neoplastic biomarker is indicative of the presence of viral-induced, cervical cancer cells in the sample. In another embodiment, the HPV-reactive reagents include a HPV E6-specific reagent and an HPV E7-specific reagent. In one embodiment, the neoplastic biomarker-reactive reagents include a p16INK4A-specific reagent and a survivin-specific reagent. In some embodiments, both the HPV-reactive and the neoplastic biomarker-reactive reagents are antibodies.

In another embodiment the system includes an electrode or micro-electrode array in place of the MEMS chip embedded within a micro-fluidic cartridge to facilitate HPV and neoplastic biomarker detection. Whereas the MEMS design carries out multiplex detection of antigens in intact cervical cells, the alternative approach detects multiple markers in cell lysates prepared from cervical specimens. In both approaches the cartridge may be integrated with a sample vial, which completes several preprocessing steps, as well as a portable reader to interpret and process signals collected from either the MEMS chip or electrode array. In the case of the MEMS-based cartridge the reader is sensing and processing an optical signal. For the alternative embodiment that includes the electrode array-based cartridge, the reader is processing a non-optical (electrical) signal. In one embodiment, design of the electrode array based cartridge leverages intellectual property of CombiMatrix Corporation described in patent application Ser. Nos. 09/944,727, 61/336,386, 10/229,775, 11/232,479 and 11/238,470, hereby incorporated by reference with respect to at least array and cartridge design parameters.

In yet another embodiment the system employs a network of carbon nanotube (CNT)-based electrodes within a microfluidic cartridge in place of the MEMS chip to capture and sense target antigens. This arrangement facilitates detection of multiple markers (DNA, RNA, protein, etc.) within the cartridge and as described earlier, is integrated with both an up-front patient sample preprocessing (PSP) unit, as well as a portable reader, which supports cartridge and PSP functions. In this presentation, CNTs coating specific electrode surfaces are further modified with capture probes for detection of specific analytes. Detection is non-optical: dependent upon the generation of electrical signals in the presence of captured analytes, with the signal being transduced through the CNT network and electrode surfaces.

### BRIEF DESCRIPTION OF THE DRAWINGS

FIG. 1 is a block diagram of an embodiment of the molecular, cervical cancer screening system. This embodiment is in the form of a MEMS-based platform.

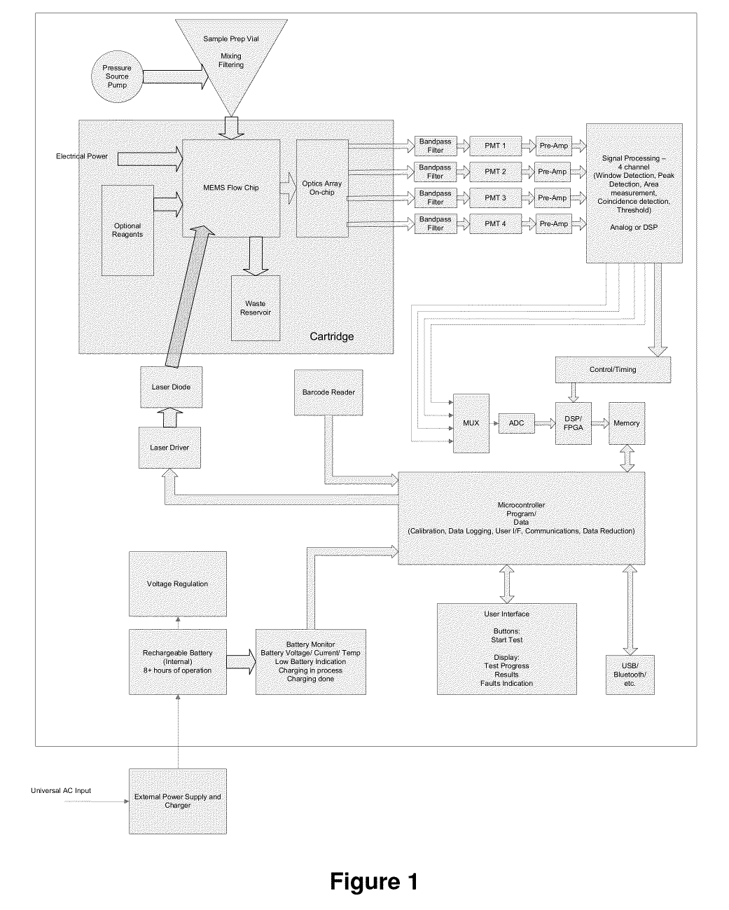

FIG. 2 depicts an embodiment of a micro-electrode array, which may be employed for single species or multiplex detection.

FIG. 3 depicts a multi-array design for multiplex or expanded uniform detection.

FIG. 4 depicts a cross-sectional view of the proposed micro-electrode array.

FIG. 5 depicts a view of a single use cartridge employing CNT-based electrodes.

FIG. 6 depicts a cross-sectional view of a CNT-based nanosensor chip.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

The present disclosure is based on a disposable cartridge for the rapid molecular detection of multiple markers associated with cervical disease in a point-of-care setting. The primary focus of the test is on the detection of HPV-associated cervical disease, by detecting the presence of HPV and downstream cervical cell changes resulting from deregulated expression of HPV and cervical cell proteins. This combined approach to assessing the disease using multiple markers that arise at different stages of disease progression results in a test that has fewer false positives and fewer false negatives. This reduction in false positive and false negative results yields a test with significantly higher sensitivity and specificity, across all patient age groups. The screening tools of the present disclosure require far less time from the patient, the physician, and laboratory testing personnel.

The cervical cancer screening tools of the present disclosure comprise reagents for detection of at least one HPV marker (e.g., viral protein or nucleic acid). In addition, the screening tools comprise reagents for detection of at least one neoplastic marker (e.g., cellular protein or nucleic acid). Detecting elevated expressions of both viral and cellular markers of cervical cancer in a single test, while optionally employing disease algorithms, increases the clinical relevance and confidence of the test. The HPV marker(s) are indicative of a persistent or transforming HPV infection. The neoplastic marker(s) are indicative of transcriptional or translational changes in the host cell that are associated with loss of cell cycle control and apoptotic processes leading to the development of cervical intraepithelial neoplasia (CIN) and ultimately to invasive cervical cancer.

A negative test for the HPV marker(s) signifies an undetectable level of HPV (no or transient infection), while a negative test for the neoplastic marker(s) signifies the absence of cervical disease. Moreover, if HPV is detected but the neoplastic marker(s) are absent, then HPV infection is likely to be inconsequential. In this instance, the patient need not be subjected to unwarranted biopsies and a presumptive cervical cancer diagnosis. Instead, such patients need only undergo periodic routine screening.

On the other hand, if the neoplastic markers are detected in the absence of HPV, then the test is indicative of cervical disease of unknown etiology, which is likely to be a rare but significant observation. Only when both the HPV and neoplastic markers of cervical cancer are present, is the test indicative of HPV-associated cervical disease (CIN 2 and above). Thus, both the positive and negative predictive value of the test is improved over prior art tests by utilization of markers of both HPV infection and neoplastic transformation in combination.

In addition to the ability of the system to detect the presence of specific biomarkers, the inclusion of controls or standards within each cartridge is critical for accurate determination of biomarker levels and changes as they relate to different stages of disease progression. Hence the system employs several controls independent of the specific mechanism for detection, e.g., flow, microelectrode or CNT-based electrode arrays. Controls include the use of non-functionalized electrodes, electrodes modified with specific classes of immunoglobulins, e.g., IgG1, IgG2, or electrodes functionalized with biomarkers for specific housekeeping proteins or nucleic acids whose levels do not fluctuate with disease. Examples of such markers may include GAPDH, actin, B-globin, etc. Controls can be used to determine specificity of binding, noise, and/or be used to normalize the level of biomarker changes detected across numerous patient samples. Control data is included in the algorithm employed for final output.

### Human Papillomavirus (HPV) Markers

Papillomaviruses are DNA viruses with a double-stranded, circular DNA genome containing a coding region for late (L) genes, a coding region for early (E) genes, and a non-coding upstream regulatory region with binding sites for the various transcription factors controlling expression of the early and late genes. Two separate open reading frames in the late gene coding region encode viral capsid proteins L1 and L2. Eight open reading frames in the early gene coding region, encode eight viral early proteins, designated E1, E2, E3, E4, E5, E6, E7, and E8. HPV can be found in cervical material in non-integrated forms (episomal), integrated forms or in mixed forms.

Increased expression of the E6 and E7 oncoproteins, due to integration of HPV DNA into the host genome or other mechanism of disrupting E2-mediated inhibition of E6 and E7 expression, induces chromosomal instability (Vinokurova et al., Cancer Research, 68:307-313, 2008). The E6 and E7 oncoproteins in turn bind to host cell proteins causing a dysregulation of cell cycle progression and proliferation (Ganguly and Parihar, J Biosci, 34:113-123, 2009). Specifically, E6 in association with host E6AP (associated protein), which has ubiquitin ligase activity, acts to ubiquinate the p53 tumor suppressor leading to its proteosomal degradation. Similarly, E7 binds to the retinoblastoma (Rb) tumor suppressor, freeing the transcription factor E2F to transactivate its targets. The E7 oncoprotein further destabilizes cell cycle control through its interaction with the cyclin-dependent kinase inhibitor protein, p21. HPV E6 and E7 oncoproteins are found to be continuously produced in transformed genital tissues. These interactions set the stage for controlling host cell proliferation and differentiation (i.e., transformation), a first step in the conversion of normal cells to pre-neoplastic cells and ultimately to the full expression of cancer malignancy.

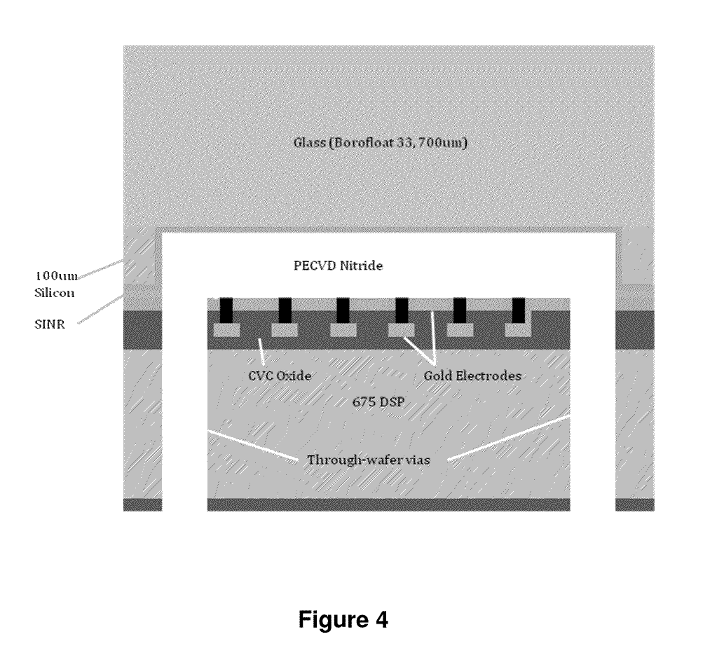

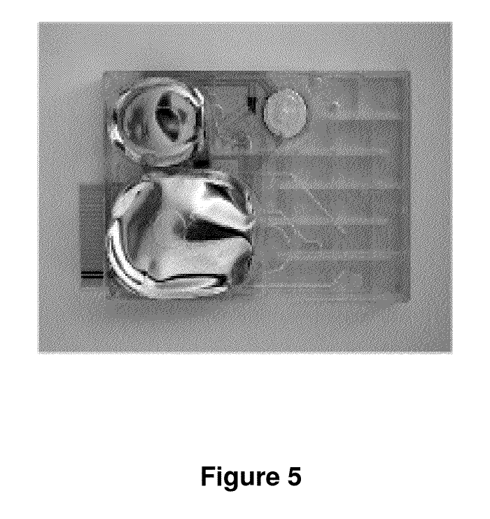

Accordingly, HPV infection may be assessed by detection of one or both of the viral E6 and E7 oncoproteins. Amino acid sequences of exemplary HPV E6 proteins and E7 proteins are disclosed in FIGS. 4A and 4B, and FIG. 5, respectively, of US 2009/0104597 of Gombrich and Golbus (herein incorporated by reference for the teaching of HPV protein sequences). In some embodiments, the reagents employed to detect the HPV marker(s) detect all HPV subtypes. In other embodiments, the reagents employed to detect the HPV marker(s) detect high risk types or only those high risk types most frequently associated with cervical cancer (e.g., HPV16, 18, 31, 33 and 45). In further embodiments, HPV presence may be confirmed by detection of additional viral markers alone (e.g., E4, E5, etc.), or in specific ratio to other viral proteins.

### Neoplastic Markers

With the advances in genomics and proteomics, a large number of cellular genes were found to be differentially expressed in cervical cancer cells in comparison to normal cervical epithelium (see, e.g., Santin et al., Virology, 331:269-291, 2005). Accordingly, a neoplastic cellular profile is assessed by detection of one or more neoplastic markers (e.g., reduced tumor suppressor levels or increased oncogene levels). In some preferred embodiments, the neoplastic marker(s) are host cell proteins that play roles in cell cycle progression or apoptosis. In exemplary embodiments, the neoplastic markers comprise p16INK4A and survivin.

In HPV-associated tumors and dysplasia, the increased expression of HPV E7 results in down-regulation of Rb, hypomethylation of the p16INK4A promoter and marked overexpression of p16INK4A. Over-expression of p16INK4a represents a marker for CIN II and CIN III, as well as cervical carcinoma (Klaes et al., Int J Cancer, 92:276-284, 2001). It is also detected in the great majority of CIN I lesions associated with high risk HPV types, while no detectable expression of p16INK4A has been found in normal cervical epithelium or inflammatory lesions. p16INK4a (also referred to herein as p16 and p16^{INK4a}) is a cyclin dependent kinase inhibitor that plays a role in regulating cell cycle progression. It is expressed as isoform 1 along with several transcript variants from the CDKN2A gene. The amino acid sequence of p16INK4A is provided as GenBank Accession No. NP_{—}000068.

Elevation of E6 following HPV integration increases expression of survivin, via inhibition of p53 mediated transcriptional regulation. Analysis of survivin levels in cervical cancer samples show a strong correlation with high risk HPV and CIN grade. Therefore, both the survivin protein and mRNA are biomarkers for cervical cancer and its precursors (Branca et al., Amer J Clin Path, 12:113-121, 2005). Survivin (also referred to as apoptosis inhibitor 4, API4, baculoviral IAP repeat-containing protein 5, and BIRCS) is a member of the inhibitor of apoptosis (IAP) gene family and as such plays a role in the control of programmed cell death. Several transcript variants of the BIRCS gene have been identified, with the most frequently occurring transcript corresponding to isoform 1, encoding a protein with the amino acid sequence provided as GenBank Accession No. NP_{—}001159.

Other biomarkers that are upregulated or downregulated in cervical cancer cells are employed in further embodiments of the present screening tools. These markers may be nucleic acid in origin (DNA, mRNA, non-coding-RNA) or protein-based markers. Options include vascular endothelial growth factors (VEGFs) (Branca et al., J Clin Path, 59:40-47, 2006), DEK (Wu et al., Pathol Int, 58:378-382, 2008), c-FLIP (Wang et al., Gynecol Oncol, 105:571-577, 2007), SIX1 or GDF15 (Wan et al., Int J Cancer, 123:32-40, 2008), as well as combinations of additional markers (Branca et al., Int J Gynecol, 27:265-273, 2008). Further options include host cell genes that are upregulated or downregulated in primary invasive cervical carcinomas (Santin et al., Virology, 331:269-291, 2005). Upregulated genes include but are not limited to: mesoderm-specific transcript, forkhead box M1, v-myb myeloblastosis viral oncogene homolog (avian)-like2 (v-Myb), minichromosome maintenance proteins 2, 4, and 5, cyclin B1, prostaglandin E synthase (PTGES), topoisomerase II alpha (TOP2A), ubiquitin-conjugating enzyme E2C, CD97 antigen, E2F transcription factor 1, and dUTP pyrophosphatase. Downregulated genes include but are not limited to: transforming growth factor beta 1, transforming growth factor alpha, CFLAR, serine proteinase inhibitors (SERPING1 and SERPINF1), cadherin 13, protease inhibitor 3, keratin 16, and tissue factor pathway inhibitor-2 (TFPI-2). Nonlimiting examples of suitable neoplastic marker(s) are listed in Table 1, or otherwise mentioned above.

TABLE 1
Neoplastic Markers

| Marker | Description |
|---|---|
| p16^{INK4A} | cyclin-dependent kinase inhibitor |
| survivin | apoptosis inhibitor 4 (AIP4) or BIRC5 |
| MCM # | Mini chromosome maintenance 2, 3, 4, 5, 6, or 7 |
| CDC6 | Cell division cycle protein 6 |
| SCC | Squamous cell carcinoma antigen |
| PCNA | Proliferating cell nuclear antigen |
| Ki-67 | Proliferation marker (MIB-1) |
| TOP2A | Topoisomerase II alpha |
| Cyclin X | Cyclins A, B, C, or D |
| CDCA 1 | Cell division cycle-associated protein 1 |
| Geminin | DNA replication inhibitor |

### Antibodies

In some embodiments, the HPV and neoplastic biomarkers are detected using marker-reactive polyclonal or monoclonal antibodies. In an exemplary embodiment, HPV markers are HPV E6 and E7, and the neoplastic markers are p16ink4a and survivin. HPV E6 and E7 are detected with antibodies cross reactive with viral antigens from multiple HPV strains. Antibodies may be purchased or licensed from a commercial source or produced in house for inclusion in the Cervical Screening platform. The neoplastic marker p16ink4a is detected with a mouse monoclonal antibody obtained from Duke University Medical Center (Durham, N.C.). Exemplary anti-p16ink4a monoclonals include JC2, JC4, and JC6 (Dai et al., Gastroenterology, 119:929-942, 2000; Furth et al., Neoplasia, 8:429-436, 2006; Gump et al., Cancer Research, 61:3863-3868, 2001; and Nielsen et al., Laboratory Investigation, 79:1137-1143, 1999). The neoplastic marker survivin may be detected with a rabbit or mouse monoclonal or polyclonal antibody obtained from a commercial source such as Cell Signaling Technology (Beverly, Mass.) or with select hybridomal clones generated in-house or through commercial vendors. The present disclosure is not limited to the detection of these biomarkers or the use of the specific antibodies listed herein for this purpose.

### Patient Population

One of the benefits of the cervical cancer screening tools of the present disclosure is that their use is not accompanied by an age recommendation. Specifically, unlike the tests of some of the prior art, the disclosed cervical cancer screening tools are appropriate for use with women of all ages, including young women who are typically excluded from use of the currently approved HPV tests that do not distinguish transient HPV infection from transforming HPV infection. Women under 30 years of age are currently an underserved population because many of them have been exposed to HPV and thus are likely to be scored as a false positive on the currently approved HPV tests.

### Other Diseases

In further embodiments, the screening tools of the present disclosure provide reagents for detection of additional markers for the detection of other infectious diseases of the cervix. The device may be further multiplexed so that the detection of multiple types of cervical infectious diseases (and cervical disease progression) and/or sexually transmitted diseases occurs on a single cartridge using the same sample. Other diseases of interest include markers for the presence of Herpes Simplex virus, Chlamydia and Neisseria gonorrhoeae, among others.

### Sample Collection

In one embodiment, test samples come (1) directly from a swab or other collection device, such as CerMed's CerMap collector, or (2) indirectly from a liquid transport/storage/processing medium. Alternatively the sample comes from an “at home” collection technique. The self-collection and self-test options open the way for women who do not or cannot have access to physicians. Expert care can be sought if warranted by the at home test.

### Sample Processing

Sample processing may include two stages: (1) initial processing after collection, hereafter referred to as preprocessing, and/or (2) processing in the device for target biomarker detection, including signal enhancement. Preprocessing may be performed prior to introducing the sample to the test device, or be performed in the test device. In an embodiment with preprocessing outside the device, the collected sample (e.g., collected using a standard cervical brush) is placed into a patient sample preprocessing (PSP) vial. The vial contains both fixed and solution-based means for sample processing including such things as filters for course and fine level filtration of cellular debris, mucous, and/or nucleic acids as well as solutions containing reagents to prepare cells for detection on either the MEMS or electrode array-based cartridge. Cells intended for processing on a MEMS-based detection cartridge will remain intact throughout the process. In embodiments utilizing a cartridge employing an electrode array-based detection, preprocessing steps will facilitate complete cervical cell lysis and recovery of the protein fraction. Vial solutions may include reagents that disrupt red-blood cells, remove cell clusters, degrade nucleic acids, inhibit protein degradation, alter membrane permeability promoting intracellular transport of antibodies to target proteins, and/or facilitate complete cervical cell disruption. Alternative approaches may also include addition of antibodies to surface markers indicative of target squamous epithelial cells, such as EpCam or cytokeratin proteins, to enrich specific cell fractions prior to analysis on the cartridge.

In an exemplary method for processing cells on the MEMS-based cartridge, a cervical cell pellet is incubated with a methanol-free paraformaldehyde in an isotonic solution (e.g., phosphate buffered saline, PBS) to fix the cells. The fixed cervical cells are then contacted with an isotonic solution containing Triton X-100 (TX100) to permeabilize the cells. The permeabilized cervical cells are subsequently incubated with an isotonic solution containing fluorochrome-conjugated antibodies to produce stained cells. Cervical cells are washed between processing steps as needed with an isotonic solution, which in some embodiments contains one or both of a blocking agent (e.g., bovine serum albumin, BSA) and a detergent (e.g., TX100). The MEMS process maintains whole cells throughout preprocessing and processing steps. In an alternative embodiment in which cervical samples are preprocessed for subsequent analysis on an electrode-array based cartridge, preservation of intact cells in the sample vial is unnecessary and instead samples are manipulated with the aim of complete lysis to release target antigens for capture and non-optical detection on the microelectrode or CNT-based electrode array.

### Preprocessing

Common steps in sample preprocessing take place independent of the detection platform, however, specific steps will be required to facilitate MEMS (whole cell), micro-electrode-based (lysate), or CNT-based electrode detection. For MEMS-based detection, cells are introduced into a vial with a dual filter arrangement that filters large and small debris/contaminants (≧100 um and ≦15 um) and traps target cells (˜30 to 70 um). The vial also contains reagents that promote RBC lysis, disrupt clusters, fix and/or permeabilize cells for subsequent introduction of antibodies to target markers. For example, red blood cells (RBC) lysis is commonly achieved by addition of a filtered lysis buffer containing 150 mM NH4Cl, 10 mM KHCO3 and 1.0 mM EDTA, pH 7.4. Fixation is preferably achieved by contacting isolated cells with methanol-free paraformaldehyde in an isotonic solution (i.e., phosphate buffered saline, PBS). Cells are permeabilized by incubation with an isotonic solution containing Triton X-100 (TX100). The washed, fixed and permeabilized cells may be incubated with antibodies directly in the vial or transferred to the cartridge where they react with fluorochrome-conjugated antibodies deposited in an initial reaction chamber.

For micro- or CNT-electrode-based detection, the vial carries out similar sample preprocessing steps required for the MEMS-based approach with the added requirement of target cell lysis prior to detection. Lysis can occur as a final stage within the vial or as an initial step on the microfluidic cartridge. Cell lysis can be achieved by a variety of common methods apparent to those familiar with the art. Lysis solutions may include addition of ionic or non-ionic detergents, protease, and/or phosphatase inhibitors, salts, buffers etc. hypotonic solutions, etc. A preferred cell lysis buffer includes a non-ionic detergent such as 1% Triton, or 1% NP-40 which is less denaturing to proteins than an ionic detergent, 20 mM Tris-HCL (pH 7.5), 150 mM NaCl, 1 mM Na2-EDTA, 1 mM EGTA, 1 mM B-glycerophosphate, 1 ug/ml leupeptin, 1 mM PMSF, and 1 mM benzamidine. The lysate is then incubated with RNase/DNase solution or filtered to remove nucleic acids prior to flow over the electrode capture array within the micro-fluidic cartridge.”

In an alternate approach for micro-electrode- or CNT-based electrode detection, the target cell population can be enriched by employing antibody(s) against surface cell proteins specific for target cells. For CIN positive cervical squamous epithelial cells these markers may include but are not limited to molecules such as Ep-CAM, or specific isoforms of cytokeratin such as C5 or C14 (Litvinov, S. V. et al., 1996 Am J. Pathol 148(3); 865-875). Antibodies may be deposited within the sample vial, presented on the surface of polystyrene or magnetic beads or upon a removable solid-state substrate with in the vial. A removable substrate can be subsequently transferred to an initial chamber within the cartridge where captured cells are lysed and target antigens flowed over the micro-electrode capture array. Alternatively, the initial chamber of the cartridge contains an array or substrate displaying antibodies for target cell capture. Preprocessed cells are flowed over the cell capture substrate within the cartridge, washed and lysed prior to flowing the lysate over an electrode-based capture array.

Antibody coated beads or free antibodies presented in the vial, or antibodies coated on available surfaces within the vial may also be used to directly complex target antigens during preprocessing steps as opposed to enriching for target cells. In this embodiment, cells are fully lysed within the sample vial and the target antigens bound to free antibodies or antibodies complexed to polystyrene or magnetic beads or vial surfaces. Target antigen-antibody complexes are washed within the vial and may be transferred to the microfluidic cartridge for further capture on the micro-electrode array. Transfer may include the direct movement of antigen-antibody-bead complexes, or free antigen. Free antigen is attained by disruption of the antibody-antigen complex following washing in the cell vial. Typical means for disruption include the use of buffers with increased salt concentration, detergents (SDS), etc. Preferred means include reversible processes such as employing buffers with increased salts, which can be removed by passing eluates through a subsequent desalting step or column.

In another embodiment target antigen enrichment in lysates can be completed prior to capture and analysis of proteins on the micro- or CNT-based electrode array. Biomarkers for HPV and cervical neoplasia exhibit similar molecular weights (−16-17 kDa). Therefore, methods that enrich for proteins of this size may improve sensitivity and reduce noise. Enrichment is achieved through any of a number of methods that reduce the fraction of proteins outside the molecular weight range of the target fraction. Protein concentration can be achieved through chromatographic means (size exclusion), filtration, or precipitation and resuspension. In the preferred approach, lysates are processed via a size exclusion method, whereby specific fractions eluting from a chromatographic column or filtered on nanopourous membrane are collected and flowed over the capture array. Size exclusion may be implemented as a final step on a lysate flowing from the sample vial to the cartridge or as an early step on the cartridge prior to micro-electrode array capture. Typically, size exclusion chromatography employs columns with considerable length to improve separation of proteins over the course of travel. To accommodate dimensional constraints of the microfluidic cartridge, size exclusion resins or membranes may be employed within serpentine or linear paths within the cartridge, extending throughout one or more layers of the cartridge to increase the length of the flow path. Alternatively, a molecular weight cut-off filter may be employed to limit the flow of certain molecular weight fractions beyond a specific region of the cartridge.

Various resins, immunoaffinity monolith columns, or nanoporous microdialysis polymer membranes may also be employed within flow paths or within microchips in the microfluidic cartridge and/or sample vial for desalting or to filter/concentrate proteins of specific molecular weight. Use of these materials would aid in adjusting reaction conditions to promote increased binding between target antigens and antibodies used to enrich target cells or capture target proteins, to promote enzymatic activity critical for processing and/or detection or to isolate specific fractions of protein in a rapid manner.

### Cartridge

In one embodiment, marker detection occurs in a disposable, point-of-care, cartridge-based system housing. This device allows the detection of up to 6 markers associated with the virus/disease at different states. The combination of markers allows a much higher level of sensitivity and specificity than available with one marker alone. The cartridge employs one or more of several detection schemes including detection of target proteins in cell lysates, detection of nucleic acids in cell lysates, detection of target proteins in permeabilized whole cell preparations, or a combination thereof. The first approach (protein or nucleic acid detection in lysates) employs specific antibodies or nucleic acid probes in a unique microfluidic cartridge employing an electrode array for biomarker capture and detection such that target proteins or nucleic acids in cell lysates originating from cervical specimens are complexed with antibodies (enzyme-linked immunosorbent assay or ELISA-type technique) or probes immobilized (microarray-type technique) on a surface-modified electrode. The second approach maintains the structural integrity of whole cells originating from cervical specimens and detects intracellular target markers using antibodies or probes in a MEMS-based cartridge facilitating flow cytometric assay.

In one embodiment, the flow cytometric assay employs one or more MEMS components. In a preferred embodiment, the MEMS components comprise any required optical, actuator and manifold layers to optimize flow performance and, preferably, a minimum of 4-color detection in a fully contained, single, disposable point-of-care cartridge. Alternatively, the cartridge can be used in conjunction with a stand-alone reader capable of delivering any required reagents and processing signals originating from biosensors on the MEMS or non-optical, electrode based microfluidic cartridge. Both the MEMS and non-optical, electrode based approaches permit detection of multiple targets in a single specimen; the former in permeabilized whole cell preparations, the latter in cell lysates. Signal processing capability may, for example, reside with the MEMS, be contained within the reader or rely on components found both on the MEMS and the reader. The cartridge is also designed to facilitate any required preprocessing steps prior to target marker detection, and may employ filtration, exploit immunological detection of surface molecules on target cells, magnetic beads, etc., to ensure entry and flow of specimen to the MEMS chip.

In the preferred embodiment the cartridge house multiple CNT-based electrode nanosensors, which comprise the array. Each nanosensor detects a specific analyte through modification of CNT surfaces with specific biomarker probes. In the CNT-configuration, nanosensors employ an electrode surface coated with a network of CNT. These can be SWNT, MWNTs, nanowires or other nanostructures, which impart improved sensitivity specificity to the electrode. CNTs may be modified with enzymes, proteins, nucleic acids, immunoglobulins, etc., providing capture and detections specificity. Several methods may be employed for the deposition of CNTs on electrode surfaces including top-down or bottom up processes, which are known to those experienced in the art.

In either the MEMS or non-optical, electrode-based approach, the test is designed to provide information to the user in a rapid, point-of-care manner, amenable to a low resource setting. This type of test could be performed in a lab. When the testing is performed in the lab, there is a physical and time separation from the patient and the answer. Thus, even if the results are negative, the patient still needs to return and re-enter the medical system. The MEMS and non-optical, electrode based approaches described herein, permit the test to be performed at the point-of-care and is especially applicable to developing countries lacking sophisticated medical and technical infrastructures. The separation of the patient, sample and test is minimal. The patient does not need to return for the results and appropriate utilization of the medical system is made possible.

A portion of the sample may be transferred directly from the sample vial to a final preprocessing stage of the test cartridge or manipulated off-cartridge to complete preprocessing steps. To prepare the sample for subsequent analysis of the target markers, the preprocessing stage is designed to clean and concentrate target cells from unwanted material, such as blood or immune cells, cellular and non-cellular debris, digestion of mucous, and polysaccharide. Suitable tools for sample preprocessing may include, but are not limited to, membrane filtration, magnetic beads or other substrates displaying specific binding moieties for the physical and immunologically-based separation and concentration of target cells. Membranes may include substrates modified with antibodies to collect target cells, or be designed based on size exclusion for filtering cells from extraneous material. Immunological enrichment steps may utilize general immunoglobulin-based capture or rely upon polystyrene or magnetic beads displaying specific reactive moieties to target cells. Preferred methods concentrate target cells or materials through capture on a surface or beads modified with antibodies or probes to cell surface or intracellular proteins, nucleic acid sequences, etc. Surface markers used to concentrate cells belong to classes of proteins that are commonly displayed on epithelial cells of the cervix (endo- and ectocervical region). Once attached to the enrichment substrate, target cells or material are washed free of unwanted components. As described above, steps may also include the use of resins or nanoporous polymer membranes to adjust buffer conditions or alter protein concentrations, promoting antigen-antibody binding.

Target cells are eluted from the preprocessing component (which may be on or off the cartridge) and introduced into the processing stage of the cartridge. Once in the device there may be additional steps that further enhance the sample to prepare it for detection of target biomarkers. Sample processing may include washing of target cells and/or incubation with reagents that: lyse or permeabilize cellular membranes, promote antigen binding to antibodies for target cell surface proteins and/or intracellular markers, inhibit protein degradation, or utilize components for physical separation of target cells/proteins/nucleic acids from unwanted materials.

In a sample destined for analysis on a micro-electrode array cartridge, cells complexed to a substrate off-cartridge, such as magnetic beads, can be collected in an initial chamber within the cartridge. Conversely, preprocessed sample may be passed over magnetic beads displaying specific binding moieties for target cell capture, which are located within an initial chamber of the cartridge. Target cells are then washed and lysed in a reduced volume of mild, detergent-based lysis buffer (present as blister pack on the cartridge or added directly by the user) designed to disrupt cellular components freeing antigens for detection in subsequent stages. The lysate is then directed to a chamber housing the micro-electrode detection array via micro fluidic channels using manually or electronically controlled valves or pumps.

Detection on a surface modified micro electrode array consists of a substrate to which capture antibodies for target biomarkers have been bound. Lysate interacts/binds with antibodies to target markers and remaining material is washed free. Arrays may be multiplexed, containing all capture antibodies in defined locations, or be individually based, such that each array is designed for detection of a specific marker. The use of multiple arrays necessitates moving the lysate over each array or moving a portion of the lysate toward a specific array. Following binding to the detection array(s), the sample is washed on the array using wash buffer present on cartridge in blister pack or supplied separately. A second antibody, specific for each antigen is delivered to each array to form a sandwich. The second antibody optionally contains a component for signal amplification or signal detection. For example, signal amplification may be accomplished by addition of an antibody with a biotin or enzymatic conjugate, while signal detection may be facilitated by an antibody conjugated to a specific enzyme or fluorescent tag. Biotin conjugated antibodies support signal amplification through the use of secondary binding tools such as streptavidin-poly-horseradish peroxidase. Similarly, antibodies conjugated to fluorescent tags can be used to detect captured antigen but necessitate the use of optics designed for to detect specific wavelengths (see reader).

### Reader

An exemplary embodiment of the integrated reader is designed to support the cartridge, providing power, reagents, signal processing, reporting and user interface options. The reader is designed as a portable, compact unit housing illumination and, if required, detection optics for marker detection; rechargeable batteries, voltage regulation and electronics; microcontroller for programming; analog and/or digital signal processing and amplification components including any required bandpass filters, photomultiplier tubes, etc., for data processing, retrieval, reporting, and calibration; and a barcode reader for sample identification. The micro-electrode or MEMS cartridge is designed to be inserted into the reader, which contains required ports and electrical connections and will facilitate sample preprocessing, marker detection and reporting of results. The reader will interpret the particular style of cartridge and specific sample ID and run any required calibration prior to sample analysis. Data will be stored on-board and is retrievable via the user interface. Optional connections for upload and download of data and/or software will allow users to interface reader with additional storage or transfer devices.

One method for biomarker detection begins by either a direct capture at the biosensing electrodes, such as the direct capture of HPV-associated nucleic acids, or proteins and/or a simple displacement assay utilizing high affinity antibodies immobilized on latex beads, polymeric membrane or metal-coated surface. Another method facilitates detection of markers within their natural surroundings through preservation of the cellular architecture. Biosensors commonly work by coupling a biomolecule to a transducer. The biomolecule may be an enzyme, an antibody, a peptide, an aptamer, and/or a nucleotide. There are many common signal transduction schemes. These transduction schemes include surface plasmon resonance spectroscopy, quartz crystal imbalance, fluorescence or near infrared spectroscopy, mass spectroscopy, electrochemical detection, feedback capacitance measurement, and others.

Two proposed methods employed in the device for detecting the HPV and neoplastic markers include 1) a scheme that result in the optical detection on a MEMS-based platform of an antigen-antibody complex in whole cells, such as through fluorescent or infrared spectroscopy, facilitated through conjugation of a reporter molecule to the employed antibodies; and 2) a non-optical scheme that employs a surface modified micro electrode array for capture and detection of antigens in cell lysates. In the former scheme, multiplex detection on a MEMS platform is achieved through the use of different fluorophores with distinct emission profiles. Fluorophores may or may not share a common excitation wavelength and illumination optics will correspond accordingly to fluor specifications. Optics on the reader incorporate one or two laser diodes, LEDs or OLEDs. Typical diodes include an INGaN/GaN, SiC diode, which emits at 380, 405, and 470 nm, and a AlGaInP/GaAs laser diode that emits at 635, 650, and 670 nm. In some embodiments, the overall emission wavelengths range from 500-750 nm when two lasers are employed or up to 700 nm when a single laser is employed.

Preferred chromofluors to be used for conjugation to primary antibodies for detection of target biomarkers are listed below in Table 2, based on a 488 nm and 640 excitation scheme. The final fluors are selected based on the preferred emission wavelengths and degree of separation/signal strength. Although in some embodiments, two excitation sources are employed, in other embodiments a single excitation source (488 nm) is utilized.

TABLE 2
Chromofluor Properties

| Laser Excite (nm) | Chromofluor | Channel Emit (nm) |
|---|---|---|
| 488 | FITC FL1 | 530 |
| 488 | GFP FL1 | 530 |
| 488 | PE FL2 | 585 |
| 488 | PI FL2 | 585 |
| 488 | PerCP FL3 | >670 |
| 488 | PerCp-Cy 5.5 FL3 | >670 |
| 488 | PE-Cy5 FL3 | >670 |
| 488 | PE-Cy7 FL3 | >670 |
| 640 | APC FL4 | 675 |

In an alternate approach to the use of chromofluors for multiplex detection, antibodies can instead be conjugated to colloidal particles or nanocrystals such as quantum dots. Quantum dots are semiconductors, often ranging in size from 5 to 50 nm, which can be tuned to emit light at specific wavelengths by tailoring the size of the particle; the larger the Q-dot the lower the energy emitted. They can be self-assembled with a core-shell structure of which the shell can be readily modified with various molecules to aid in solubility or direct binding as in the case of an antibody. Q-dots are brighter, more stable and do not suffer from the level of photobleaching that can occur with the use of chemical dyes.

In the micro electrode approach employing a non-optical means of detection, antigen binding is detected through current generated in the presence of an enzyme-antibody conjugate and appropriate chemistry. Perturbation of the dielectric constant or changes in resistance detected on an absorbing, or conductive layer around a captured molecule could be the result of a captured antigen and/or of a captured nucleic acid. An amplification scheme may or may not need to be employed. Amplification of signal can be accomplished by several methods such as through the use of secondary antibodies conjugated to specific enzymes for colorimetric enhancement, polymer formation, generation of substrates for target signal enhancement, or electronics and processing for signal gain or noise depletion.

In one example the biosensor device simultaneously captures and detects the presence of the HPV and neoplastic markers (nucleic acid or protein) found in cell lysates. In an alternate approach, such as that used in flow cytometry, detection of cells containing antibodies bound to target proteins is accomplished through changes in fluorescence at specific wavelengths corresponding to particular fluorophores conjugated to marker-specific antibodies.

### Flow Cytometric Platform

In the flow-based embodiment, the cervical screen reader is designed for use with dedicated cervical screen cartridges. On board optics are provided for multiplex illumination and sensing for detection of targeted biomarkers. The reader has a data interface standard HL7 for hospital communication protocol and possesses a calibration chip/process for daily calibration. A low cost bar-code reader is provided to read labels attached to the MEMs cartridge. The User Interface (UI) and output is adapted for positive or negative detection of target biomarkers with algorithm(s) to assess signal strength relative to background and to provide information to the user. An error indicator for processing failure is also provided. The reader is further equipped with on board memory for daily data download, and is CE and UL certified. In preferred embodiments, the reader is portable, battery powered instrument that runs on standard alkaline or rechargeable batteries. Through its interface with the test cartridge, the reader unit provides power to support operation of the test cartridge if an auxillary power source is needed. In some embodiments, the reader comprises an integrated sample pre-processing component, for removal of undesirable material (e.g., blood cells, mucous, etc.). In these embodiments, the reader further comprises reagent and wash buffer storage and disposal features.

In a preferred MEMS embodiment, the test cartridge comprises a MEMS chip embedded within a cartridge (laminate, injection molded or otherwise) that provides an inexpensive, flow-cytometric platform for detection of cervical cancer cells in solution. In some embodiments, the cartridge comprises a chip, a sample input reservoir, and a sample collection reservoir, with the sample collection reservoir disposed to collect processed material for each chip. In some embodiments, the cartridge comprises a mechanism to distinguish debris from whole cells for use with an event counter. A preferred MEMS chip provides the following properties. The preferred chip can analyze a minimum of 50,000 cells in 15 minutes. It further provides multiplex fluorescent detection capability within a single cell to permit the simultaneous measurement of multiple biomarkers of viral or mammalian cell origin. It is also a gating tool to distinguish between debris and whole cells, facilitating processing of a defined number of target cells. The test cartridge is designed as a disposable, self-contained unit, providing a device for sample analysis and collection of processed materials. The primary sample pre-preparation is done off-cartridge. The test cartridge comprises a sample identification function (e.g., bar-code) and is integratable with the reader unit (e.g., combinable elements such as electrical circuits, fluid paths, inputs/outputs, etc.). The target sensitivity is 90% or greater for CIN2 (cervical intraepithelial neoplasia, grade 2), with a target specificity of 90% or greater.

Exemplary MEMS designs, which may be readily adapted to provide a flow cytometric platform for the screens described herein, may be found, for example, in the following patents. U.S. Pat. No. 7,264,972 describes an actuator-based cell sorting MEMS platform that incorporates fluorescence-based detection and sorting. Other MEMS chips, which may find use, or may be adapted for use, in the methods and systems described herein, are provided in U.S. Pat. Nos. 6,838,056, 7,220,594, and 7,229,838. Similarly, other MEMS chips known in the art and suitable for use as flow cytometric platforms may be used.

### Micro-Electrode Platforms

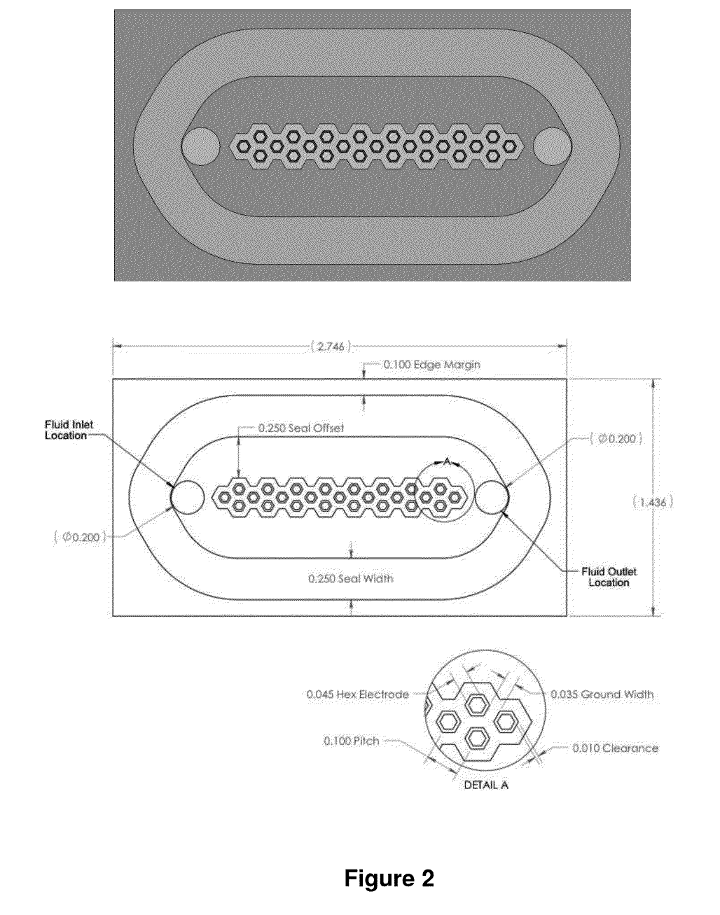

In the preferred embodiment, the design of the micro fluidic cartridge employs an array(s) of surface modified electrodes rather then a MEMS chip. These electrodes may be based on the use of a network of CNT-based nanosensors, or PCB-based microelectrode arrays. These approaches facilitate multiplex detection of antigens in cell lysates opposed to whole cells. A simple, PCB-based microelectrode array may be comprised of approximately 25-27 individual electrodes (e.g., FIG. 2 depicts an array with 25 electrodes). The surface of each electrode is covered with a thin layer of conductive polymer embedded with antibodies to specific biomarkers. Each electrode may display the same or distinct antibody; replicate electrodes provide improved sampling and reproducibility for data analysis. Exemplary design and detection processes, which are hereby incorporated by reference, for the original high density arrays are reported in U.S. patent application Ser. Nos. 09/944,727, 61/336,386, 10/229,775, 11/232,479 and 11/238,470. While the original platform employed a high density array containing over 12K platinum electrodes produced by a CMOS-based manufacturing process, the current embodiment may employ a small number of gold micro electrodes manufactured using wafer-based processes, compatible with MEMS manufacturing processes. For example, the number of electrodes in the array may be 50, 45, 40, 35, 30, 25, 20, 15, or 10 electrodes. In addition to gold, other electrode materials known in the art may be used. A cross sectional illustration of the manufactured chip is provided in FIG. 4. The preferred embodiment employs a network of CNT-based nanosensors with a single microfluidic cartridge to capture and detect analytes in solution. CNT-based electrodes provide a highly sensitive and pliable network for detection of analytes. Preferred embodiment incorporates designs and functionalization processes described in filing 20120018301, and patents referenced therein.

To facilitate antigen detection, the surface of each micro-electrode is modified by depositing a thin layer of an organic conductive polymer into which specific antibodies to HPV and cervical cancer biomarkers are adsorbed. Conductive polymers are characterized by molecules whose backbone contains aromatic cycles (e.g., polyaniline, polypyrrole, poly(fluorene)s, polypyrenes, polyazulenes or polynapthalenes), double bonds (e.g. poly(acetylene)s or both (e.g., poly(p-phenylene vinylene). Their conductivity results from their carbon structure, which displays conjugated bonds (pi-bonds (sp2) orbitals), with (pi)-orbital delocalization. Preferred coatings for array electrodes utilize the aromatic cycle-containing polypyrrole. Antibodies adsorbed to the polymer-coated electrodes serve as the target antigen capture locale. Polymer deposition is controlled by individually addressing each electrode with a brief, defined voltage or current for a specified period. The strength and time of activation result in differing thickness of the polymer being deposited and altering the sensitivity of the platform. The preferred embodiment deposits conductive polymer using a constant voltage ranging from 0.7 and 1.9 V for 5 s, however different antibodies may exhibit improved sensitivity under other deposition voltages.

In CNT-based electrode embodiments, the electrode surface is coated with a network of CNTs, which improves electrode sensitivity and imparts a means for selective analyte detection. In this case, the CNT-array is further modified by embedding or covalently attaching specific agents for biomarker detection directly to the CNT surface as opposed to the electrode surface. Agents may include the antibodies described herein, or other capture agents such as nucleic acids, peptide nucleic acids, aptamers, proteins, etc. As with the PCB-based microelectrode arrays, the CNT-based arrays, the network of CNT deposited on the surface may be further modified with specific polymer coatings to impart additional properties of conductance, impedance, etc, or to form a link to attachment of specific biomarker probes.

Target antigens present in cell lysates prepared from cervical specimens are captured on the micro-or CNT electrode array modified with specific antibodies. Occupied electrodes are detected by sandwich addition of HRP-conjugated antibody to the primary antigen bound on the electrode. Electron flow produced as a result of the enzymatic redox reaction with HRP in the presence of appropriate chemistry (TMB), are transduced through the array and recorded by the Reader. Signal amplification can be accomplished electronically or through use of secondary conjugates such as anti-mouse or rabbit antibodies labeled with biotin for reaction with streptavidin-poly HRP conjugates. As with MEMS-based detection, the Reader employs a signal processing algorithm for quantitative signal assessment and results are provided through a user interface component of the reader.

An illustration of one possible embodiment of a micro-electrode array is presented in FIG. 2. The design incorporates 25 gold electrodes arranged on a silicon substrate. Measurements are in millimeters.

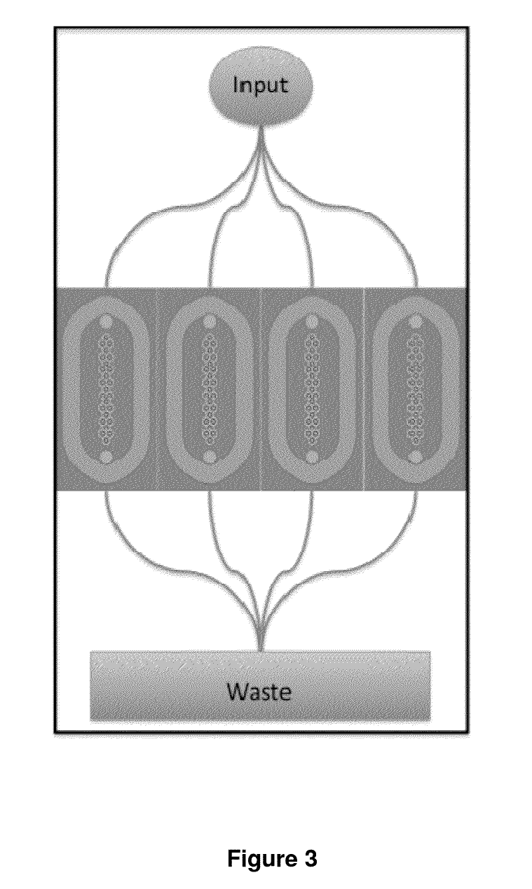

The array may function as a uniform antibody array whereby all 25 electrodes capture the same antigen, or as a mixed antibody array in which individual electrodes within a single array are embedded with distinct antibodies for capture of specific antigens. Multiplex detection can be achieved by use of a mixed multi-antibody array or by using multiple, uniform antibody arrays in parallel or series, each capturing a specific antigen. An example of a multi-array design for multiplex is provided FIG. 3. Array dimensions are equivalent to those presented above.

In one embodiment, the array chip is manufactured using a complementary metal-oxide-semiconductor (CMOS) process. In an alternative embodiment, the array employs manufacturing processes compatible with MEMS manufacturing and may be built on a silicon-nitride substrate with glass cover. A cross sectional illustration of the proposed chip is presented in FIG. 4.

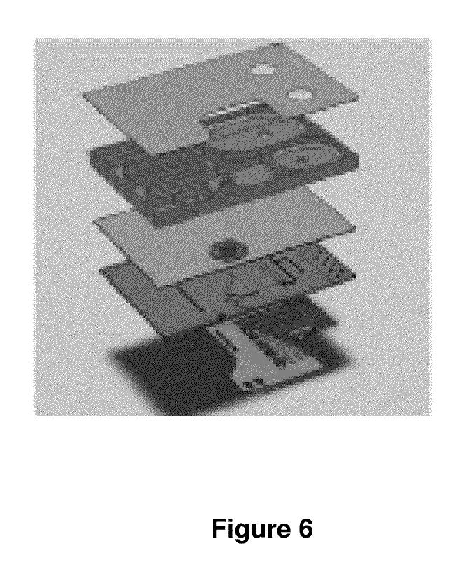

In the preferred embodiment the array is comprised of a network of CNT-based nanosensors. Each nanosensor is functionalize with a specific and unique capture probe to facilitate capture and detection of a distinct analyte. Presentation of multiple nanosensors on the same microfluidic cartridge imparts multiplex capability. Representative images of nanosensor chips and cartridges is provided in FIGS. 5 and 6.

Additionally, MEMS components could also be integrated with the micro-electrode array to provide for various functions. For example, a MEMS device could cause the sample to flow over the micro-electrode array.

1. A system for detecting human papillomavarius (HPV)-induced cervical cancer, the system comprising:
a cartridge, the cartridge comprising:
a sample collection area for receiving a biological sample;
an HPV biomarker antibody, wherein when an HPV biomarker binds with the HPV biomarker antibody, a detectable first signal results;
a cervical cancer biomarker antibody, wherein when a cervical cancer biomarker binds with the cervical cancer biomarker antibody, a detectable second signal results; and
a detection chamber for reacting the biological sample with the HPV biomarker antibody and the cervical cancer biomarker antibody; and
a reader, the reader comprising:
an interface for receiving the cartridge; and
a circuit for measuring the first and second signals, wherein the first signal is indicative of the presence of the HPV biomarker in the biological sample and the second signal is indicative of the presence of the cervical cell biomarker in the biological sample.

2. The system of claim 1 wherein:
the cartridge further comprises:
a micro-electrode array having a plurality of electrodes for detecting the first and second signals;
an organic conductive polymer layer coating the micro-electrode array; and
a cartridge interface on the cartridge, wherein the cartridge interface is electrically connected to the micro-electrode array;
the HPV biomarker antibody is embedded in the organic conductive polymer layer and the first signal is electrical;
the cervical cancer biomarker antibody is embedded in the organic conductive polymer layer and the second signal is electrical;
the detection chamber is configured to house the micro-electrode array and to bring the biological sample in contact with the organic conductive polymer layer coating the micro-electrode array; and
the reader further comprises:
a reader interface for electrically connecting to the cartridge through the cartridge interface.

3. The system of claim 1 wherein:
the cartridge further comprises:
a MEMS chip for processing the biological sample into a processed sample by mixing the biological sample with the HPV biomarker antibody and the cervical cancer biomarker antibody;
the detection chamber is further configured to receive an excitation light to illuminate the processed sample and allow transmission of optical responses of the processed sample;
a first reporter molecule is conjugated to the HPV biomarker antibody and the first signal is a first optical response of the first reporter molecule to the excitation light;
a second reporter molecule is conjugated to a cervical cancer biomarker antibody and the second signal is a second optical response of the second reporter molecule to the excitation light;
the reader further comprises:
a first light source configured to provide the excitation light to the detection chamber, wherein the emission profile of the first reporter molecule in response to the excitation light is different than the emission profile of the second reporter molecule in response to the excitation light; and
the circuit for measuring the first and second signals further comprises detection optics for producing a first electrical signal from the first signal and a second electrical signal from the second signal.

4. The system of claim 1, wherein the HPV biomarker is a first protein and the cervical cancer biomarker is a second protein.

5. The system of claim 1, wherein the micro-electrode array is metal on a silicon substrate.

6. The system of claim 1, wherein the micro-electrode array is an array of a plurality of micro-electrode arrays.

7. The system of claim 6, wherein the cartridge is configured so that each micro-electrode can be operated independently of each other.

8. The system of claim 1, wherein the conductive polymer layer on some of the plurality of electrodes of the micro-electrode array is embedded with the HPV biomarker antibody but not the cervical cancer biomarker and the conductive polymer layer on some of the plurality of electrodes of the micro-electrode array are embedded with the cervical cancer biomarkers antibody but not the HPV biomarker antibody.

9. The system of claim 1, the cartridge further comprising:
a micro-fluidic channel for transporting the biological sample to the detection chamber.

10. The system of claim 1, the cartridge further comprising:
a reagent reservoir containing a reagent reactive with the HPV biomarker antibody or the cervical cancer biomarker antibody.

11. The system of claim 1, wherein the reagent is horseradish peroxidase conjugated to a second HPV biomarker antibody or the reagent is horseradish peroxidase conjugated to a second cervical cancer biomarker and oxidation of the horseradish peroxidase results in the first and second signals.

12. The system of claim 3, wherein the first reporter molecules is a chromofluor, fluorophore, or quantum dot and the second reporter molecules is a chromofluor, fluorophore, or quantum dot.

13. The system of claim 3, the reader further comprising:
a second light source configured to provide a second excitation light to the detection chamber, wherein the emission profile of the first reporter molecule to the second excitation light is different than the emission profile of the second reporter molecule in response to the second excitation light.

14. The system of claim 1, wherein the HPV biomarker is HPV E6 or HPV E7 and the cervical cancer biomarker is p16ink4a or survivin.

15. The system of claim 1, wherein the cartridge is a single-use cartridge.

16. The system of claim 1, where the sample collection area is in detection chamber.

17. The system of claim 1, the system further comprising:
a vial for preprocessing the biological sample prior to applying the biological sample to the cartridge.

18. The system of claim 17, the vial further comprising:
a first filter for removing debris or contaminants smaller than target cells; and
a second filter for removing debris or contaminants larger than target cells.

19. The system of claim 17, the vial further comprising:
an antibody for enrichment of target cell fractions.

20. The system of claim 17, the vial further comprising:
a removable solid-state substrate for transferring the biological sample from the vial to the sample collection area on the cartridge.

21. A system of claim 1, wherein the cartridge further comprises:
a plurality of CNT-based nanosensors for capture and non-optical detection of specific analytes.

22. The system of claim 21, wherein the CNT-based nanosensors are functionalized with agents for biomarker capture.

23. The system of claim 21, where the CNT-based nanosensors are functionalized with polymer coatings.

24. The system of claim 21, wherein the CNT-based nanosensors comprise single-walled nanotubes (SWNTs) and/or multi-walled nanotubes (MWNTs).
